(19) United States
(12) Patent Application Publication
(10) Pub. No.: US 2020/0105389 A1
(43) Pub. Date: Apr. 2, 2020

(54) MINING SENTIMENTS BY BIO-SENSING TO IMPROVE PERFORMANCE

(71) Applicants: Aashka Garg, Santa Clara, CA (US); Aniket Singhai, Cupertino, CA (US); Avika Garg, Santa Clara, CA (US); Priya Jain, Los Altos, CA (US)

(72) Inventors: Aashka Garg, Santa Clara, CA (US); Aniket Singhai, Cupertino, CA (US); Avika Garg, Santa Clara, CA (US); Priya Jain, Los Altos, CA (US)

(21) Appl. No.: 16/586,611

(22) Filed: Sep. 27, 2019

Related U.S. Application Data
(60) Provisional application No. 62/738,757, filed on Sep. 28, 2018.

(51) Int. Cl. G16H 15/00; G16H 10/65; G16H 20/70; G16H 20/30; G16H 40/67; G06K 9/00; H04L 29/08; G10L 25/63; G10L 15/22; G10L 15/26; G09B 19/00; G09B 7/00; A61B 5/16; A61B 5/024; A61B 5/08; A61B 5/00

(52) U.S. Cl. CPC G16H 15/00; G16H 70/00; G16H 20/70; G16H 20/30; G16H 40/67; G06K 9/00335; H04L 67/10; G10L 25/63; G10L 15/22; G10L 15/265; G09B 19/0038; G09B 7/00; A61B 5/165; A61B 5/024; A61B 5/0816; A61B 5/7239; A61B 5/7257; G16H 10/65

(57) ABSTRACT

The disclosed embodiments relate to bio-sensing to detect various emotions and improve feedback and targeted assistance for users performing or learning to perform tasks. In one example embodiment, a method may include detecting a biomarker of a user using a biosensor associated with the user, determining a psychophysiological state of the user based on the biomarker detected by the biosensor, and providing feedback to the user via a user interface of a social networking application that links a set of users including the user to improve performance of the user in executing a task based on pre-defined metrics.

### CROSS-REFERENCE TO RELATED APPLICATIONS

This application claims the benefit of U.S. Patent Provisional Application No. 62/738,757, filed Sep. 28, 2018, titled MINING SENTIMENTS BY BIO-SENSING TO IMPROVE COGNITIVE PERFORMANCE, which is incorporated herein by reference in its entirety.

### BACKGROUND

The present disclosure generally relates to an application that improves feedback and targeted assistance for users performing or learning to perform tasks. The disclosed embodiments may be implemented in a variety of circumstances. For example, a task may include completing a homework assignment, engaging in a sports activity, public speaking, interviewing or acting.

A common problem faced by many, especially those in the younger age groups, is to seek or receive very targeted help or feedback when & where substantial performance enhancement may be required. That is, the cases that will be significantly helped by an improved psychophysiological concordance. Such challenges may arise in the context of preparing for a speech or interview, completing a difficult homework assignment, finding a motivation to exert oneself when tired or depressed, faced with a sports challenge or a social challenge, such as bullying, etc.

Accordingly, the disclosed embodiments implement bio-sensing to detect various emotions and improve feedback and targeted assistance for users performing or learning to perform tasks.

The claimed subject matter is not limited to embodiments that solve any disadvantages or that operate only in environments such as those described above. This background is only provided to illustrate examples of where the present disclosure may be utilized.

### SUMMARY

The present disclosure generally relates to bio-sensing to detect various emotions and improve feedback and targeted assistance for users performing or learning to perform tasks.

In one non-limiting embodiment, a method may include detecting a biomarker of a user using a biosensor associated with the user, determining a psychophysiological state of the user based on the biomarker detected by the biosensor, and providing feedback to the user via a user interface of a social networking application that links a set of users including the first user to improve performance of the first user in executing a task based on pre-defined metrics. The pre-defined metrics may be known to the users of the linked set of users to provide feedback or assistance using the social networking application. In some embodiments, the metric indicating success may be well-defined, and/or well expounded, in advance. The task may include completing a homework assignment, engaging in a sports activity, public speaking, interviewing or acting.

In some aspects, the biosensor may be included in a wearable network-connected device associated with the user. The biomarker may include heart rate or respiration of the user. The method may include analyzing the biomarker using non-linear analysis, a PNNx technique, a Root Mean Square of the Successive Differences (RMSSD) technique or Fast Fourier Transforms of respiration. The method may include determining a gap between the psychophysiological state of the user and a desired psychophysiological state.

In some aspects, the task performed by the user may be a homework assignment, and the method may include continuously monitoring stress levels of the user during performance of the homework assignment using the biomarker detected by the biosensor. The method may include displaying a stress level and a level of completion of the homework assignment corresponding to the first user, to a second user using a second user interface of the application.

The method may include displaying one or more of: availability to help of a second user to the first user via the user interface of the application; comprehension of a topic of the first user to a second user via a second user interface of the application; a suggestion of a study partner based on study habits and patterns; and a grade impact scale indicating an impact of the homework assignment on an overall grade of the user.

The task performed by the user may be a sports technique, and the method may include detecting a position and a movement of a portion of the user's body using a sensor; obtaining a desired position and desired movement corresponding to the sports technique; and comparing the desired position and the desired movement with the detected position and the detected movement of the portion of the user's body. In some aspects, the desired position and desired movement may correspond to a position and a movement of a portion of a professional sports athlete's body. Providing feedback to the user may be based on differences between the desired position and the desired movement with the detected position and the detected movement of the portion of the user's body. In some aspects, the method may include analyzing a mindset of the user based on the biomarker of the user, analyzing comprehension of feedback received by the user; and displaying the comprehension or the mindset of the user to a second user via a second user interface of the application.

The task performed by the user may be an interview, and the method may include detecting a set of characteristics of the user performing the interview; and comparing the detected set of characteristics with a desired set of characteristics. The set of characteristics may include one or more of: emotions of the user, posture of the user, tone of the user, and content of the user's responses. In some aspects, providing feedback to the user may be based on differences between the detected set of characteristics and the desired set of characteristics of the interview.

The task performed by the user may be a speech, and the method may include obtaining text of the speech including annotations corresponding to desired emotions to be emoted in the speech; obtaining a video of the user performing the speech using a camera associated with the user; detecting a set of characteristics of the user performing the speech; and comparing the detected set of characteristics with a desired set of characteristics. The set of characteristics may include one or more of: emotions of the user, tone of the user, hand gestures of the user, and body language of the user. In some aspects, providing feedback to the user may be based on differences between the detected set of characteristics and the desired set of characteristics of the speech.

The task performed by the user may be a theater scene, and the method may include obtaining script of the theater scene including annotations corresponding to desired emotions to be emoted in the theater scene; obtaining a video of the user performing the theater scene using a camera associated with the user; detecting a set of characteristics of the user performing the theater scene; and comparing the detected set of characteristics with a desired set of characteristics. The set of characteristics may include one or more of: emotions of the user, tone of the user, hand gestures of the user, and body language of the user. In some aspects, providing feedback to the user may be based on differences between the detected set of characteristics and the desired set of characteristics of the theater scene.

In some aspects, the psychophysiological states may correspond to a diagram of a circumplex model of emotion.

This Summary introduces a selection of concepts in a simplified form that are further described below in the Detailed Description. This Summary is not intended to identify key features or essential characteristics of the claimed subject matter, nor is it intended to be used as an aid in determining the scope of the claimed subject matter.

### BRIEF DESCRIPTION OF THE DRAWINGS

FIG. 1 is a diagram of a circumplex model of emotion.

FIG. 2 is a block diagram of an example of a system for improving feedback and targeted assistance for users.

FIG. 3 is a block diagram of another example of a system for improving feedback and targeted assistance for users.

FIGS. 4A-4E are flow diagrams of example methods to provide feedback and targeted assistance for users.

FIG. 5 is a diagrammatic representation of a machine in the example form of a computing device within which a set of instructions, for causing the machine to perform any one or more of the methods discussed herein, may be executed.

### DETAILED DESCRIPTION

Reference will be made to the drawings and specific language will be used to describe various aspects of the disclosure. Using the drawings and description in this manner should not be construed as limiting its scope. Additional aspects may be apparent in light of the disclosure, including the claims, or may be learned by practice.

The disclosed embodiments general relate to an application that improves feedback and targeted assistance for users performing or learning to perform tasks. The disclosed embodiments may be implemented in a variety of circumstances. For example, a task may include completing a homework assignment, engaging in a sports activity, public speaking, interviewing or acting.

The disclosed embodiments implement bio-sensing to detect various emotions (or psychophysiological states) and improve feedback and targeted assistance for users performing or learning to perform tasks. In particular, disclosed embodiments implement advanced bio-sensing to detect a variety of emotions or psychophysiological states very accurately as well as with very short latency. Accordingly, the disclosed embodiments may be used to provide feedback when there is a gap between the optimal psychophysiological state of the user with his, actual, current state. In contrast, typical systems may detect a very limited set of emotions, in any, and that at a very high latency.

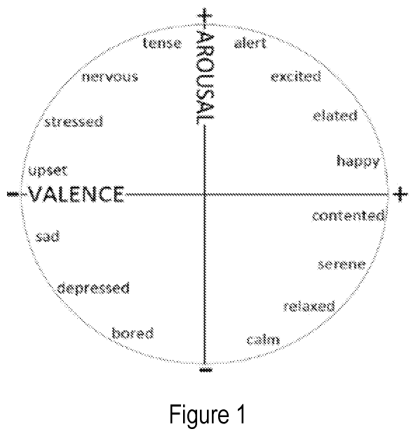

FIG. 1 is a diagram of a circumplex model of emotion. The illustrated model was developed by James Russell and may be referred to as Russell's circumplex diagram. This model suggests that emotions are distributed in a two-dimensional circular space, including arousal and valence dimensions. Arousal represents the vertical axis and valence represents the horizontal axis, while the center of the circle represents a neutral valence and a medium level of arousal. In this model, emotional states may be represented at any level of valence and arousal, or at a neutral level of one or both of these factors. Emotions may also be referred to as psychophysiological states.

Existing approaches are generally limited in their ability to provide feedback and prepare a user for a task. Although some approaches may use information about a person's mental state, such approaches typically rely on techniques that are effective only in measuring quadrants II and IV. That is, when the valence is positive with the associated arousal being low, or the valence is negative with the associated arousal being high. This unfortunately limits capturing information regarding when a user is feeling elated or is bored. In addition, current feedback applications do not use the information about a person's mental state in conjunction with providing help from a peer to peer network for a task.

Accordingly, the present disclosure includes embodiments that may thoroughly evaluate a user's mental state, using, for example, biosensors or other sensors to obtain information for a user. Such configurations may be implemented to obtain assistance to perform or learn to perform a task. For example, such aspects may be used to obtain help for a homework assignment. Such embodiments may identify when a user is stuck in a panic state, for example, while doing homework, and provide assistance in such circumstances. In another example, the disclosed embodiments may be used to prepare a user using a database of emotions synchronized in time to prepare for a task, such as an important speech. In yet another example, the disclosed embodiments may be used to master a skill or technique in a sport, for example, by providing feedback and/or constructive criticism to a user.

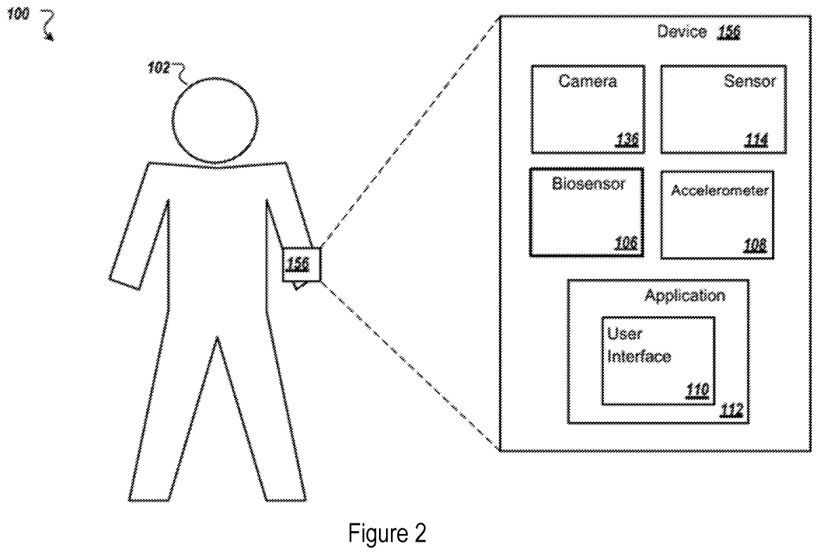

FIG. 2 is a block diagram of an example of a system 100 for improving feedback and targeted assistance for users. The system 100 may include a user 102 and a device 156. The user 102 may include any individual, person, student, athlete or other entity seeking to accomplish a task or learn to perform a task. The device 156 may be a computing device, a mobile device or other suitable device associated with the user 102.

In the system 100, emotions and/or other information regarding the user 102 may be detected based on digital signals received from biosensors or other types of sensors, such as a biosensor 106 and/or a sensor 114. The biosensor 106 and/or the sensor 114 may include an electrical potential sensor, a respiratory sensor, an ultrasound sensor, a PPG sensor, a camera, a mechanical expansion sensor, an acoustic sensor, or another suitable sensor that may receive data representative of the biological process or biomarker.

In some embodiments, the biosensor 106 and/or the sensor 114 may be included as part of the device 156. The device 156 may be a mobile phone or a wearable device, such as a network-connected smart watch, headphones, or other suitable device. In other configurations, the biosensor 106 and/or the sensor 114 may be a dedicated network-connected devices, for example, a skin-mounted sensor to monitor the user. Such devices may be coupled to the device 156, for example, via a wireless network, wireless connection, or wired coupling.

The digital signals and/or emotions may be derived from a biological process or a biomarker of a user 102 using the biosensor 106 and/or the sensor 114. For example, a biological process may include a heartbeat, respiration, a chest expansion, or another biological process. In another example, biomarkers may include heart rate variability, variation of cardiac parameters due to respiratory sinus arrhythmia (RSA), change in skin conductance (e.g., galvanic skin response), and/or energy in various EEG bands (such as an alpha band). In addition, the disclosed embodiments may measure the entire range of emotions described and represented in the circumplex model of emotion of FIG. 1. In particular, the disclosed embodiments may determine which specific emotion and intensity of the emotion associated with the user 102, as described in the circumplex model of emotion of FIG. 1, based on the digital signals derived from the sensors in response to changes in biological process and/or biomarkers. The digital signals may be obtained from the sensors and/or computed in real time or substantially real time.

In some embodiments, the system 100 may include techniques for heart rate analysis such as non-linear techniques for analyzing heart rate dynamics that are capable to provide a feedback in 10 seconds. Other embodiments may include PNNx techniques that by their very nature can provide analysis of very short time series. PNNx may include a time domain measure of respiration. Further embodiments may include Root Mean Square of the Successive Differences (RMSSD) techniques. Additionally, embodiments include technique-based analysis of frequency content of the respiration wave form when analyzed using, for example, Fast Fourier Transforms (FFT), although other configurations may be implemented.

In some aspects, the biosensors 106 may be included in a wearable network-connected device associated with the user 102. The biomarker may include any psychophysiological measure that is captured directly or indirectly with biosensors 106 such as heart rate, blood pressure, respiratory parameters, galvanic skin response, oxygen saturation, cardiac output, pulse-wave-transit-time (PWTT) or EEG spectrum of the user 102. The biosensors 106 may measure biological changes that respond to human stress and mood. The computational methods used to identify mood may include analyzing the shift in autonomic nervous system (ANS), substantial facets of which can be captured by various sensors; for example, computing heart rate variability (HRV) using heart rate time-series using non-linear analysis, a PNNx technique, Root Mean Square of the Successive Differences (RMSSD) technique, or SDNN, etc. Similarly, Fast Fourier Transforms of respiration waveform may be used. Determining a shift of ANS towards sympathetic nervous system (SNS) or parasympathetic nervous system (PSNS) using one or more of sensors that respond may be implemented. As long as the heart rate data captured can be analyzed at 1 KHz (directly or by interpolation), or can show variation in stress levels after completion of inspiration or expiration phases of ventilation, the analytical methods may implemented. In some aspects, the latency of the methods to calculate mood must not be such that changes in stress level cannot be detected in a few seconds (or, the time taken for a full ventilation).

The system 100 may include a camera 136 to record video and/or still images of the user 102. In some embodiments, the camera 136 may be included as part of the device 156. For example, the camera 136 may be included in a mobile phone or a wearable device, such as a network-connected smart watch, headphones, or other suitable device. In other configurations, the camera 136 may be a dedicated device, for example, a network-connected or wired camera to provide video or still images associated with the user 102. In such configurations, the camera 136 may be coupled to the device 156, for example, via a wireless network, wireless connection, or wired coupling.

Additionally or alternatively, the system 100 may include an accelerometer 108 to measure acceleration and/or position of the user 102. In some configurations, the accelerometer 108 may measure acceleration and/or position of a portion of the user 102, such as a specific body part of the user (hand, foot, leg, arm, torso, etc.), although other configurations may be implemented. In some embodiments, the accelerometer 108 may be included as part of the device 156. For example, the accelerometer 108 may be included in a mobile phone or a wearable device, such as a network-connected smart watch, headphones, or other suitable device. In other configurations, the accelerometer 108 may be a dedicated device, for example, a network-connected or wired accelerometer to provide acceleration and/or position data associated with the user 102, or a portion of the user, such as a body part. In such configurations, the accelerometer 108 may be coupled to the device 156, for example, via a wireless network, wireless connection, or wired coupling.

The system 100 may include an application 112, such as a mobile application on the device 156. The application 112 may improve feedback and targeted assistance for the user 102 performing or learning to perform tasks. For example, the application 112 may improve feedback and targeted assistance the user 102 completing a homework assignment, engaging in a sports activity, public speaking or interviewing.

The application 112 may thoroughly evaluate the mental state of the user 102, using, for example, the biosensor 106 and/or the sensor 114 to obtain information about the user 102. Such configurations may be implemented to obtain assistance to perform or learn to perform a task. For example, such aspects may be used to obtain help for a homework assignment when the user 102 is having difficulty or in a panic state. In another example, the application 112 may be used to prepare the user 102 using a database of emotions synchronized in time to prepare for a task, such as an important speech. In yet another example, application 112 may be used by the user 102 to master a skill or technique in a sport, for example, by providing feedback and/or constructive criticism to the user 102.

Although in some embodiments the application 112 is a mobile application loaded on the device 156, in other embodiments the application 112 may be included on other devices. For example, the application 112 may be loaded on a personal computer or desktop computer, or other computing devices such as a tablet or network connected smart device. In further embodiments, the application 112 may be loaded on a server and provided to the user 102 via the device 156 using a wireless or wired network.

The application 112 may include a user interface 110 to interact with the user 102. In some configurations, the user interface 110 may include one or more pieces of hardware configured to receive input from and/or provide output to the user 102. In some embodiments, the user interface 110 may include one or more of a speaker, a microphone, a display, a keyboard, a touch screen, or a holographic projection, among other hardware devices.

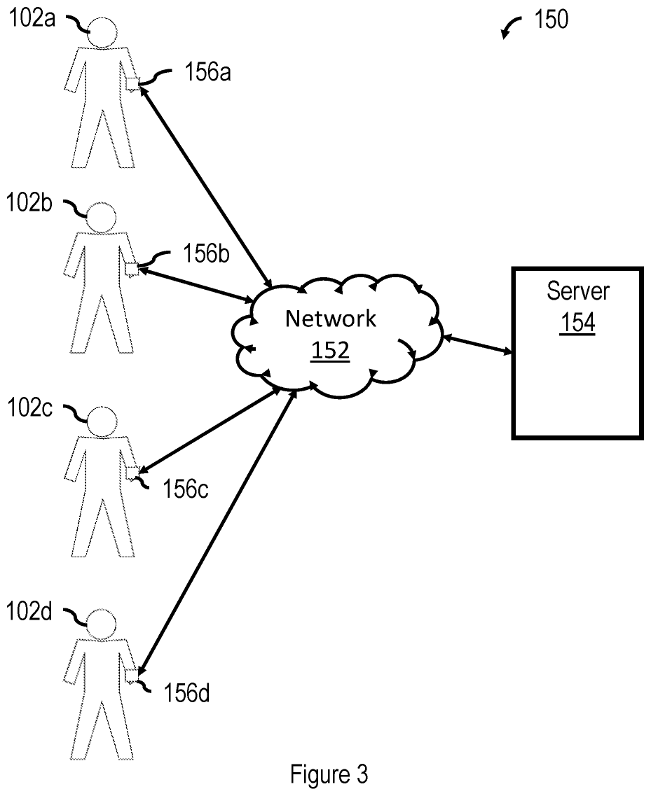

FIG. 3 is a block diagram of another example of a system 150 for improving feedback and targeted assistance for users. As illustrated, the system 150 may include multiple users 102a-d associated with corresponding devices 156a-d. The users 102a-d may correspond to the user 102 of FIG. 2 and may include any suitable aspects described therein. Further, the devices 156a-d may correspond to the device 156 of FIG. 2 and may include any suitable aspects described therein.

The devices 156a-d may be communicatively coupled to a network 152. The network 152 may include any network configured for communication of signals between the devices 156a-d, a server 154, and/or other devices. For example, the network 152 may include a local area network (LAN), a wide area network (WAN) (e.g., the Internet), and/or other interconnected data paths across which multiple devices may communicate. In some embodiments, the network 152 may include a peer-to-peer network. The network 152 may also be coupled to or include portions of a telecommunications network that may enable communication of data in a variety of different communication protocols. In some embodiments, the network 152 includes or is configured to include a BLUETOOTH® communication network, a Wi-Fi communication network, a ZigBee communication network, an extensible messaging and presence protocol (XMPP) communication network, a cellular communications network, any similar communication networks, or any combination thereof for sending and receiving data. The data communicated in the network 152 may include data communicated via short messaging service (SMS), multimedia messaging service (MMS), hypertext transfer protocol (HTTP), direct data connection, wireless application protocol (WAP), e-mail, or any other protocol that may be implemented with the devices 156a-d.

The network 152 may provide peer-to-peer communication functionality for the devices 156a-d to communicate to one another, and as such, may facilitate the users 102a-d to communicate with one another, as will be described in further detail below.

As mentioned above, the application 112 of FIG. 2 may be loaded on the devices 156a-d. In other embodiments the application 112 may be included on the server 154. In such configurations, the application 112 may be loaded on the server 154 and provided to the users 102a-d via the devices 156a-d via the network 152. Thus, the server 154 may provide at least a portion of the functionality of the application 112. In further aspects, the server 154 and the devices 156a-d may act in conjunction to provide the functionality of the application 112. In such configurations, both the server 154 and the devices 156a-d may include hardware and/or software to provide the functionality of the application 112.

As mentioned, the application 112 may improve feedback and targeted assistance for the user 102 performing or learning to perform tasks. In particular, the application 112 may improve feedback and targeted assistance the user 102 completing a homework assignment, engaging in a sports activity, public speaking or interviewing. Such aspects will now be described in further detail.

### Homework Help

In one example aspect, the application 112 may improve feedback and targeted assistance for the user 102 (which may be a student) completing a homework assignment. Social anxiety is a common problem faced among students, and in particular teenagers. The worry of being misjudged and evaluated negatively instills fear within a person, which prevents them from asking others for help and therefore prevents them from improving their mastery and understanding of content. Accordingly, the application 112 may facilitate easing the stigma associated with requesting and receiving help associated with homework.

In particular, the application 112 may implement biosensing technology to monitor a student's stress levels and homework completion and display this to other students and teachers. In such aspects, the biosensor 106 and/or the sensor 114 may be used to monitor a student's stress levels. The students and/or the teacher may correspond to the users 102a-d and may be associated with the devices 156a-d. These students and teachers can help the struggling student by chatting with them and explaining the homework to decrease their stress levels. Accordingly, the application 112 may include a chat functionality to the permit the users 102a-d to communicate with one another via the network 152 using the devices 156a-d

Thus, the application 112 may include a homework completion bar, a sensor determined stress value, a reward system, a peer to peer connection (e.g., via the network 152), a chat client, a general discussion forum, a grade impact slider, the ability to get assistance from teachers, and a teacher moderator option. Students and/or teachers may set the level that homework is completed for a give student using the homework completion bar. In some aspects, the students and/or teachers may select an importance of a homework assignment, based on, for example, the impact the homework assignment has on an overall grade in the class, or other factors as may be applicable.

In some embodiments, the application 112 may have one or more display bars, which may be generated and displayed to the users 102a-d via the devices 156a-d. The display bars may display or otherwise indicate various attributes and/or factors associated with a specific one of the users 102a-d, such as stress levels, level of homework completion or other characteristics. A student may input how much of the homework they have completed, their opinion on the complexity of the assignment, and the biosensing technology may update the metrics of priority, urgency, and impact of homework assistance and calibrate the stress level accordingly.

The application 112 may also display or otherwise indicate a student's availability, which may indicate whether or not a peer (e.g., another student or another user 102a-d) is available for help and/or if they should not be disturbed even though they are available. The application 112 may permit students to ask other students for help, for example, using the chat functionality or other suitable interface of the application 112. The application 112 may indicate students that should not be disturbed. In some aspects, the application 112 may permit messages to be sent with an indication of urgency, such as an urgent stamp on them, which may indicate to others that a student really needs help. Based on availability, if a person understands the concept that the homework assignment is on, then they can offer assistance to their peers (e.g., other students or other users 102a-d). When the student finishes the assignment, they may be given a reward, or they may receive points directly via the application 112. In some aspects, the points received via the application 112 may be redeemed for a gift card or other suitable reward.

In another aspect, the application 112 may include the ability to discover other students who have the same class and/or teacher. For example, the application 112 may include groups of students and/or users 102a-d that are part of the same class or involved in the same homework assignment. The application 112 may also include the ability to discover students who have similar classes. For example, a student taking trigonometry can be assisted by some user who is enrolled in trigonometry or a more advanced math class. The application 112 may permit students and/or users 102a-d to chat and/or video chat to one another (e.g., via the chat interface) about a homework assignment. This will expand their connections with their peers (e.g., other users 102a-d), as well as give them advice and strategies on how to complete difficult questions on the homework assignment. In some aspects, the application 112 may include a group chat functionality. The group chat functionality may utilize a microphone and camera (such as the camera 136 and/or a microphone of the user interface 110), which may be used to host study sessions with peers online.

In yet another aspect, the application 112 may include a general discussion forum, which may permit students to discuss their classes, homework assignments, tests, preparation strategy etc. The application 112 may include a grade impact slider, which teachers may use to display how much a certain assignment will be worth in their class and therefore lower a student's stress level. Teachers may also be available to give assistance to their students via the application 112 (e.g., via the user interface 110). In some aspects, teachers may be given the power to moderate students' posts including delete those that are inappropriate to the discussion in the application 112.

In some embodiments, the application 112 may have a peer to peer connection and/or communication channel. The application 112 may suggest friends based on a student's work habits and patterns by accumulating user statistics. The usage patterns that the system will analyze may include whether a peer works on assignments at similar times with similar paces, whether they typically finish assignments at the same time, and whether they often respond to the questions a potential friend asks. The application 112 may then recommend students and/or users 102a-d that may be good potential study partners, for example, based on work schedules or other factors. The application 112 may permit students to communicate with peers without using other platforms. The peer helping the given student may also receive a score from 1 to 10 of the utility of the help provided. A peer may have an option of having his score be displayed against his name. Likewise, a student seeking help may choose to be assigned a gratitude score, for example, from 1 to 10 and may have the option of having a gratitude score displayed against his name. This may allow the network to grow based upon the personality of the user and provides incentive to its members for being considerate of each other.

### Sports Technique Help

In another example aspect, the application 112 may improve feedback and targeted assistance for the user 102 (which may be an athlete) to improve or master a sports technique. To fully master a sport or specific sports technique, feedback and constructive criticism may be provided to an athlete to improve their skills. Accordingly, the application 112 may include a virtual coach to create a more personalized and accurate analysis of movement and technique for a user 102. To implement this analysis, an inertial sensor may be incorporated to determine the position of the user 102, and other relevant spatial coordinates and metrics. Additionally or alternatively, a biosensor may be implemented to determine the emotions of the user 102, such as the biosensor 106.

Position of the user 102 (and/or a portion thereof, such as a body part) may be determined using the accelerometer 108. The accelerometer 108 may have the ability to detect movement and/or direction. The accelerometer 108 may be used to recognize patterns, duration, and intensity, which may be determined, calculated and/or recorded using the application 112. Once movement is recognized (e.g., by the application 112), the athlete's movement may be compared to a signature. The athlete's movement may indicate the athlete's form in performing an athletic skill or move, in part or in whole. The signature may indicate a desired or ideal form for an athletic skill or move in the sport. The signature may be determined by collecting data (e.g., using accelerometers or other sensors) from professional athletes or others with desirable or ideal form for a given skill or move. The application 112 may determine (or may be used to determine) differences between the ideal form and a given user's form. Additionally or alternatively, the application 112 may suggest or recommend improvements based on the determined differences, or the application 112 may be used to suggest or recommend improvements based on the determined differences. For example, if a swimmer wants to perfect a flipturn, which is a somersault in the water to change direction, their movement may be analyzed by the application 112 based on data from the biosensor 106, the sensor 114 and/or the accelerometer 108, differences may be determined, which may be used to make certain changes in leg or arm position.

Emotion may also impact the mindset and/or performance of an athlete or user 102. The biosensor 106 may be used to determine an athlete's feelings and/or emotions. Having a positive mindset may be an important part of performance and success as an athlete. There may be a wide range of mindsets and the biosensor 106 may be used to make changes to improve an athlete's mindset. For example, the gap between different high-performing swimmers may be very small, so small that the only thing really distinguishing each swimmer in their performance is their mindset. Having a good mindset may be used by a swimmer to work hard and not quit until their goal is reached.

In another aspect, the application 112 may use emotion detected by the biosensor 106 to detect understanding of a certain action or idea. If a coach (who may be one of the users 102a-d) is attempting to explain a concept to an athlete, they may want to know that the athlete understands them. If an athlete is struggling to comprehend a complex idea, the coach may be able to guide them with more accuracy if they could determine their level of understanding. Accordingly, the biosensor 106 may be used to track the athlete's emotions and detect whether an athlete understands a specific concept. The application 112 may permit a coach to access this information, and this information may be used to improve teaching effectiveness.

The application 112 may also be used to improve feedback for sports skills. In particular, the application 112 may be used to correct and improve form and technique of athletes (who may be one of the users 102a-d) performing an athletic move. For example, the application 112 may use signals from accelerometers to correct and improve form and technique of an athlete engaged in an athletic move. This may be especially useful to improve athletic skills for very advanced users.

In some embodiments, very short snippets of an athletic move may be obtained by the sensor 114 and/or accelerometer 108, for example, using millisecond accuracy, or better, with components identified in all three dimensions (X, Y, Z) of movement. The accelerometer 108 may detect location, movement, speed, and/or other components of the user 102. The application 112 may then compare these components to the corresponding components of a desired or ideal form. Such aspects may be used to provide recommendations for improvement or otherwise improving the skills of the user 102.

### Speaking Task Help

In another example aspect, the application 112 may improve feedback and targeted assistance for the user 102 to improve or master speaking tasks such as interviewing, speeches or theater performance. In some embodiments, the application 112 may permit the user 102 to select a mode for interview, speech, and/or theater, for example, via the user interface 110. For speaking task help, application 112 may be augmented by natural language processing methods that can detect emotions by analyzing the speech.

The application 112 may be used to grade the user 102 based on various criteria, such as confidence, clarity, posture, body gestures, tone, emotion and/or memorization. The criteria may be determined, for example, based on data from the camera 136, the sensor 114, the biosensors 106 that can detect, for example, heart rate and/or mood, and/or the accelerometer 108. In some aspects, the application 112 may grade the user performing a speaking task, for example, based on a scale. In one example, the scale may be a 1 to 10 scale.

For interview and speech mode, the criteria graded for a speaking task may include one or more of: 1) confidence: confidence may be indicated by minimal to no hesitation and expressing ideas confidently; 2) clarity: presenting the user's ideas and answers with clarity 3) posture: indicated by the user sitting up straight with the correct posture implies attentiveness and shows the interviewer you are listening to what is being communicated; 4) body gestures: while speaking, using proper professional hand and body gestures; 5) tone: while speaking, a user should vary their tone to ensure your audience stays interested; 6) emotion (for speech and theater): present varying emotion so the audience can connect with your thoughts; and/or 7) memorization: the ability to memorize text based on a script.

In some aspects, the user 102 may annotate desired and/or required emotions on a script for the speaking task. The application 112 may score or judge the emotions based on the annotations on a script.

The some aspects, the user may be graded on a scale indicating confidence level selected by the user. For example, the scale may include low, medium and high. The scale may be selected based on the character and nature of the user's role in the speaking task, such as performance. For example, if the user is performing a role of someone who is clumsy and has low self-esteem, the user may select “low” on the confidence level. In some configurations, the application 112 may have an option to change setting This app will automatically set all levels as high, but there will be the option to change this in settings.

In some aspects, the application 112 have different modes for Interview Practice, Speech Practice, and Theater Practice modes.

In Interview Practice mode, a user may practice interview skills by doing a mock interview with the application 112 asking you mock interview questions. For example, the interview questions may be indicated via the user interface 110 and the user 102 may provide answers via the camera 136. The application 112 may judge the interview based using the biosensors 106, the sensor 114 and/or the accelerometer 108, and provide feedback on the user's interview skills based on qualitative features such as range of emotion, content, posture, tone, and other important aspects necessary for a good interview. The application 112 may categorize aspects of the interview or speech for different occasions such as college interview, job interview, school club interview, etc. In some aspects, the application 112 may include a desired standard or criteria for an interview or speech.

In speech practice mode, a user may practice their speech skills by presenting a speech in front of the camera 136. The user 102 may submit a script of a speech to the application 112 with annotated parts indicating where the user needs to convey a certain emotion. The application 112 may use the biosensor 106, the sensor 114 and/or the accelerometer 108 to judge the speech and provide feedback based on qualitative speaking skills such as voice tone, hand gestures, body language, range of emotions, and more.

In theater mode, a user may perform an act or scene in front of the camera 136. The user 102 may submit a script and/or may input comments to identify the emotion that should be displayed at each part to the application 112. The application 112 may use the biosensor 106, the sensor 114 and/or the accelerometer 108 to detect each emotion displayed by the user and each part corresponding to the script. The application 112 may provide feedback indicating how well the user displayed specific emotions.

In some aspects, the application 112 may be used by other users such as coaches or teachers to provide feedback regarding speaking tasks using the application 112, based on information from the application 112 regarding the speaking tasks.

Modifications, additions, or omissions may be made to the system 100, the system 150 and/or the application 112 without departing from the scope of the present disclosure. Moreover, the separation of various components in the embodiments described herein is not meant to indicate that the separation occurs in all embodiments. In addition, it may be understood with the benefit of this disclosure that the described components may be integrated together in a single component or separated into multiple components.

### Methods of Providing Feedback and Targeted Assistance

Methods of providing feedback and targeted assistance for users performing or learning to perform tasks, for example, using the system 100, the system 150 and/or the application 112, will be described in further detail below.

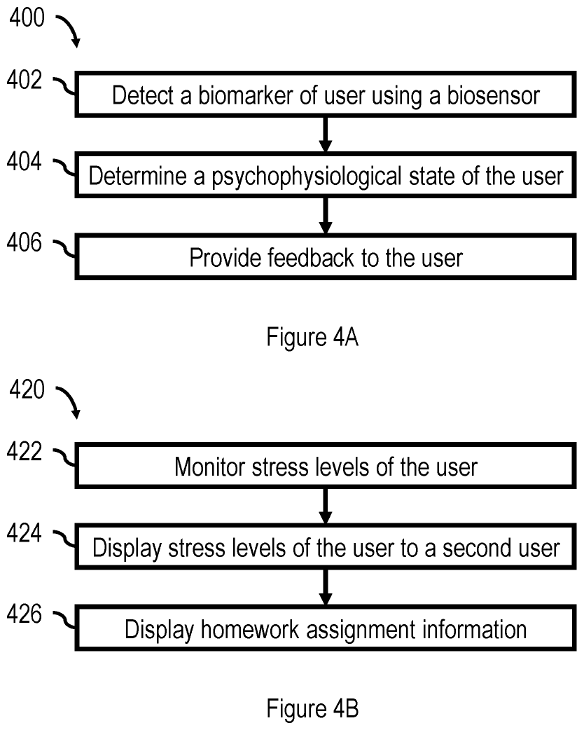

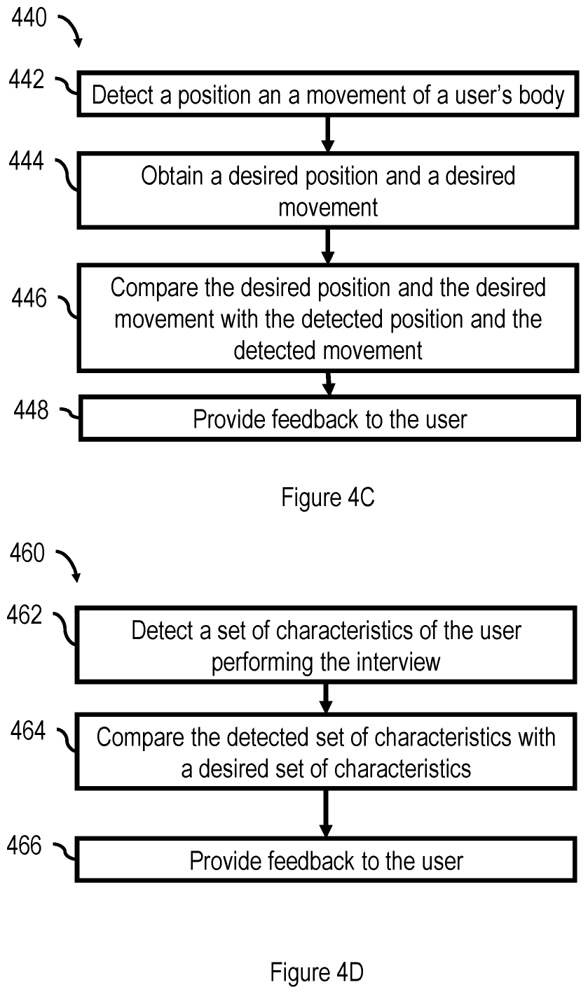

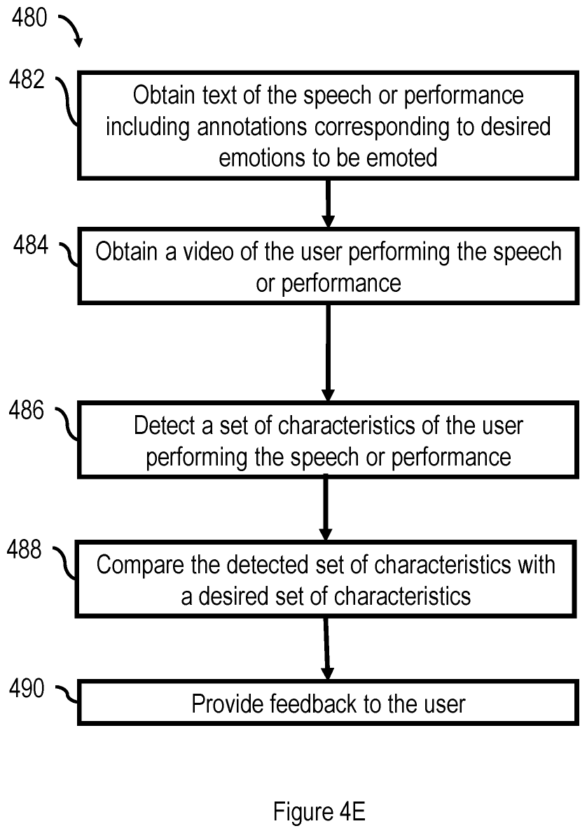

FIGS. 4A-4E are flow diagrams of example methods to provide feedback and targeted assistance for users. The methods described may implement bio-sensing to detect various emotions and improve feedback and targeted assistance for users performing or learning to perform tasks. The task may include completing a homework assignment, engaging in a sports activity, public speaking, interviewing or acting.

The methods may be performed by processing logic that may include hardware (circuitry, dedicated logic, etc.), software (such as is run on a general purpose computer system or a dedicated machine), or a combination of both, which processing logic may be included in the devices 156 and/or the server 154 of FIGS. 2-3, or another computer system or device. However, another system, or combination of systems, may be used to perform the described methods.

For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

FIG. 4A is a flow diagram of an example method 400 to provide feedback and targeted assistance. For example, the method 400 may be performed by the devices 156 and/or the server 154 of FIGS. 2-3, or a combination thereof. The method 400 may be performed to provide feedback and targeted assistance for the user 102.

The method 400 may begin at block 402, in which a biomarker of a user may be detected using a biosensor. The biosensor may be associated with a user. For example, the biosensor may be positioned on or attached to the user. In some configurations, the biosensor may be or may be included in a wearable network-connected device associated with the user. For example, the biosensor may be included in a smartwatch or skin-mounted device, or the like. The biosensor may correspond to the biosensor 106 of FIG. 2, and may include any aspects described. In some aspects, the biomarker may include a heart rate or respiration of the user, any of the other biomarkers described herein, or any other suitable biomarkers.

At block 404, a psychophysiological state or emotion of the user may be determined. The psychophysiological state or emotion of the user may be determined based on the biomarker detected by the biosensor. In some aspects, the psychophysiological state or emotion of the user may be determined by the application 112 of FIGS. 2-3. In some embodiments, the method 400 may include analyzing the biomarker using non-linear analysis, a PNNx technique, a Root Mean Square of the Successive Differences (RMSSD) technique or Fast Fourier Transforms of respiration. In some aspects, the psychophysiological states or emotions may correspond to a diagram of a circumplex model of emotion, such as the diagram illustrated in FIG. 1.

Furthermore, in some embodiments, the method 400 may include determining a gap between the psychophysiological state or emotion of the user and a desired psychophysiological state or emotion.

At block 406, feedback may be provided to the user. The feedback may be provided to the user via a user interface of an application to improve performance of the user in performing a task. For example, the feedback may be provided to the user via the user interface 110 of the application 112 of FIG. 2. The feedback may be provided to the user via a user interface of a social networking application that links a set of users including the user to improve performance of the user in executing a task based on pre-defined metrics. The pre-defined metrics may be known to the users of the linked set of users to provide feedback or assistance using the social networking application. The feedback may be used by the user to improve performance in performing the task. In some embodiments, the task may include completing a homework assignment, engaging in a sports activity, public speaking, interviewing, acting, or any suitable combination thereof. In some embodiments, the method 400 may include a variety of tasks, all the tasks described herein, or a combination thereof.

In some aspects, the task performed by the user may be a homework assignment, and the method 400 may include continuously monitoring stress levels of the user during performance of the homework assignment using the biomarker detected by the biosensor. The method 400 may include displaying a stress level and a level of completion of the homework assignment corresponding to the first user, to a second user using a second user interface of the application. Additionally or alternatively, the method 400 may include displaying one or more of: availability to help of a second user to the first user via the user interface of the application; comprehension of a topic of the first user to a second user via a second user interface of the application; a suggestion of a study partner based on study habits and patterns; and a grade impact scale indicating an impact of the homework assignment on an overall grade of the user.

In some aspects, the task performed by the user may be a sports technique, and the method may include detecting a position and a movement of a portion of the user's body using a sensor; obtaining a desired position and desired movement corresponding to the sports technique; and comparing the desired position and the desired movement with the detected position and the detected movement of the portion of the user's body. In some aspects, the desired position and desired movement may correspond to a position and a movement of a portion of a professional sports athlete's body. Providing feedback to the user may be based on differences between the desired position and the desired movement with the detected position and the detected movement of the portion of the user's body. In some aspects, the method may include analyzing a mindset of the user based on the biomarker of the user, analyzing comprehension of feedback received by the user; and displaying the comprehension or the mindset of the user to a second user via a second user interface of the application.

In some aspects, the task performed by the user may be an interview, and the method 400 may include detecting a set of characteristics of the user performing the interview; and comparing the detected set of characteristics with a desired set of characteristics. The set of characteristics may include one or more of: emotions of the user, posture of the user, tone of the user, and content of the user's responses. In some aspects, providing feedback to the user may be based on differences between the detected set of characteristics and the desired set of characteristics of the interview.

In some aspects, the task performed by the user may be a speech, and the method 400 may include obtaining text of the speech including annotations corresponding to desired emotions to be emoted in the speech; obtaining a video of the user performing the speech using a camera associated with the user; detecting a set of characteristics of the user performing the speech; and comparing the detected set of characteristics with a desired set of characteristics. The set of characteristics may include one or more of: emotions of the user, tone of the user, hand gestures of the user, and body language of the user. In some aspects, providing feedback to the user may be based on differences between the detected set of characteristics and the desired set of characteristics of the speech.

In some aspects, the task performed by the user may be a theater scene, and the method 400 may include obtaining script of the theater scene including annotations corresponding to desired emotions to be emoted in the theater scene; obtaining a video of the user performing the theater scene using a camera associated with the user; detecting a set of characteristics of the user performing the theater scene; and comparing the detected set of characteristics and their intensity and relative proportion with a desired set of characteristics, their intensity and relation proportion. The set of characteristics may include one or more of: emotions of the user, tone of the user, hand gestures of the user, and body language of the user. In some aspects, providing feedback to the user may be based on differences between the detected set of characteristics and the desired set of characteristics of the theater scene.

FIG. 4B is a flow diagram of an example method 420 to provide feedback and targeted assistance for a homework assignment. For example, the method 420 may be performed by the devices 156 and/or the server 154 of FIGS. 2-3, or a combination thereof. The method 420 may be performed to provide feedback and targeted assistance for the user 102, who may be a student or another user performing a homework assignment.

The method 420 may begin at block 422, in which stress levels of the user may be monitored. The stress levels may be monitored using biomarker detected by a biosensor. In some embodiments, the stress levels may be continuously or periodically detected by the biosensor. Accordingly, the biosensor may continuously or periodically detect the biomarkers.

The biosensor may be associated with the user. For example, the biosensor may be positioned on or attached to the user. In some configurations, the biosensor may be or may be included in a wearable network-connected device associated with the user. For example, the biosensor may be included in a smartwatch or skin-mounted device, or the like. The biosensor may correspond to the biosensor 106 of FIG. 2, and may include any aspects described. In some aspects, the biomarker may include a heart rate or respiration of the user, any of the other biomarkers described herein, or any other suitable biomarkers.

At block 424, the stress levels of the user may be displayed to a second user. The second user may be a teacher or instructor, for example, of the first user. The stress levels may correspond to the first user. The stress levels may be displayed to the second user via user interface of the application that corresponds to the second user. The second user may use the application to continuously or periodically monitor stress levels of the first user, and provide assistance or feedback in completing the homework assignment based at least in part on the stress levels.

The method 420 may include displaying a level of completion of the homework assignment corresponding to the first user, to the second user (e.g., teacher or instructor) using the interface of the application that corresponds to the second user. The second user may provide assistance or feedback in completing the homework assignment based at least in part on the level of completion.

At block 426, other homework assignment information may be displayed. The homework assignment information may include availability to help of a second user to the first user. The availability may be displayed to the first user via the user interface of the application. The homework assignment information may include comprehension of a topic by the first user. The comprehension may be displayed to a second user via an interface of the application corresponding to the second user. The homework assignment information may include a suggestion of a study partner based on study habits and patterns. The suggestion may be provided to the first user via the user interface of the application to assist in finding a study partner. The homework assignment information may include a grade impact scale indicating an impact of the homework assignment on an overall grade of the user. The grade impact scale may be displayed to the user via the user interface of the application.

FIG. 4C is a flow diagram of an example method 440 to provide feedback and targeted assistance for a sports technique. For example, the method 440 may be performed by the devices 156 and/or the server 154 of FIGS. 2-3, or a combination thereof. The method 420 may be performed to provide feedback and targeted assistance for the user 102, who may be an athlete or another user learning or perfecting a sports technique.

The method 440 may begin at block 442, in which a position and a movement of a portion of the user's body may be detected using a sensor. For example, the position and the movement may be detected using the accelerometer 108 of FIG. 2, or another suitable sensor.

At block 444, a desired position and desired movement may be obtained. In particular, the desired position and the desired movement may correspond to the sports technique being performed by the user. In some aspects, the desired position and desired movement corresponds to a position and a movement of a portion of a professional sports athlete's body.

At block 446, the desired position and the desired movement may be compared with the detected position and the detected movement of the portion of the user's body. In some configurations, the comparison may be performed by the application, for example, at a device corresponding to the user or at a server.

At block 448, feedback may be provided to the user. In some configurations, the feedback may be provided to the user via the user interface of the application. The feedback may be based on differences between the desired position and the desired movement with the detected position and the detected movement of the portion of the user's body.

In some embodiments, the method 440 may include analyzing a mindset of the user based on the biomarker of the user, analyzing comprehension of feedback received by the user, and/or displaying the comprehension or the mindset of the user to a second user via a second user interface of the application.

FIG. 4D is a flow diagram of an example method 460 to provide feedback and targeted assistance for an interview. For example, the method 460 may be performed by the devices 156 and/or the server 154 of FIGS. 2-3, or a combination thereof. The method 460 may be performed to provide feedback and targeted assistance for the user 102, who may be preparing for an interview.

The method 460 may begin at block 462, in which a set of characteristics of the user performing the interview may be detected. In some configurations, the set of characteristics may be detected by the camera 136, the sensor 114, the biosensor 114, and/or the accelerometer 108 of FIG. 2, or any suitable combination thereof.

At block 464, the detected set of characteristics may be compared with a desired set of characteristics. In some configurations, the comparison may be performed by the application, for example, at a device corresponding to the user or at a server. In some aspects, the set of characteristics may include one or more of: emotions of the user, tone of the user, hand gestures of the user, and body language of the user.

At block 466, feedback may be provided to the user. In some configurations, the feedback may be provided to the user via the user interface of the application. The feedback may be based on differences between the detected set of characteristics and the desired set of characteristics.

FIG. 4E is a flow diagram of an example method 480 to provide feedback and targeted assistance for a speech or performance. For example, the method 480 may be performed by the devices 156 and/or the server 154 of FIGS. 2-3, or a combination thereof. The method 480 may be performed to provide feedback and targeted assistance for the user 102, who may be preparing for a speech or performance

The method 480 may begin at block 482, in which text of the speech or performance including annotations corresponding to desired emotions to be emoted in the speech or performance may be obtained.

At block 484, a video of the user performing the speech or performance may be obtained. In some configurations, the video of the user performing the speech or performance may be obtained using a camera associated with the user, such as the camera 136 of FIG. 2.

At block 486, a set of characteristics of the user performing the speech or performance may be detected. In some configurations, the set of characteristics may be detected by the camera 136, the sensor 114, the biosensor 114, and/or the accelerometer 108 of FIG. 2, or any suitable combination thereof.

In some aspects, the set of characteristics may include one or more of: emotions of the user, tone of the user, hand gestures of the user, and body language of the user. In another aspect, the set of characteristics may include one or more of emotions of the user, tone of the user, hand gestures of the user, and body language of the user.

At block 488, the detected set of characteristics may be compared with a desired set of characteristics. In some configurations, the comparison may be performed by the application, for example, at a device corresponding to the user or at a server.

At block 490, feedback may be provided to the user. In some configurations, the feedback may be provided to the user via the user interface of the application. The feedback may be based on differences between the detected set of characteristics and the desired set of characteristics.

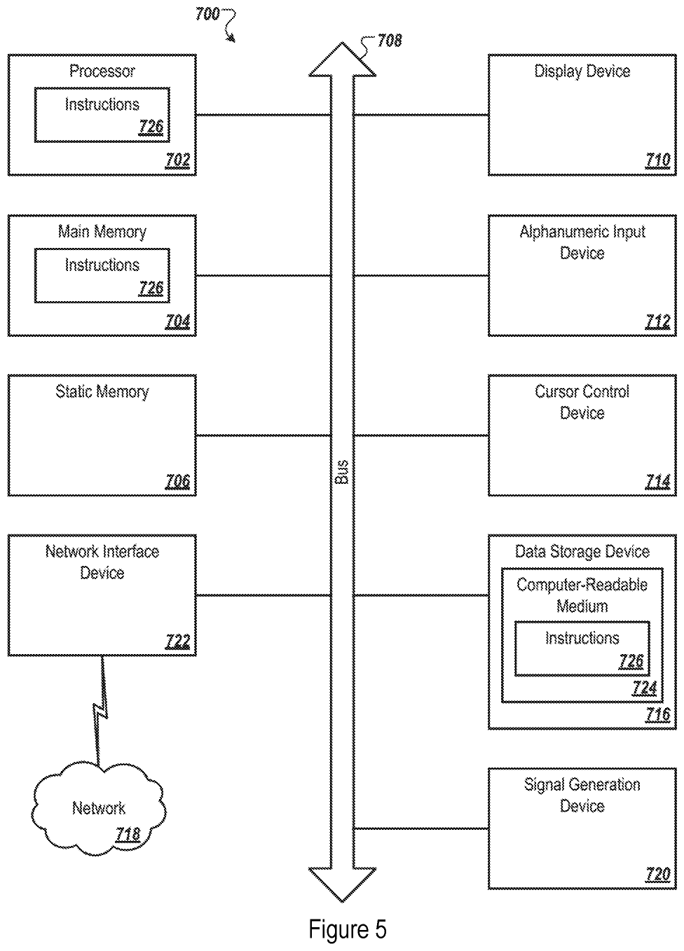

FIG. 5 illustrates a diagrammatic representation of a machine in the example form of a computing device 700 within which a set of instructions, for causing the machine to perform any one or more of the methods discussed herein, may be executed. The computing device 700 may include a mobile phone, a smart phone, a netbook computer, a rackmount server, a router computer, a server computer, a personal computer, a mainframe computer, a laptop computer, a tablet computer, a desktop computer etc., within which a set of instructions, for causing the machine to perform any one or more of the methods discussed herein, may be executed. In alternative embodiments, the machine may be connected (e.g., networked) to other machines in a LAN, an intranet, an extranet, or the Internet. The machine may operate in the capacity of a server machine in client-server network environment. The machine may include a personal computer (PC), a set-top box (STB), a server, a network router, switch or bridge, or any machine capable of executing a set of instructions (sequential or otherwise) that specify actions to be taken by that machine. Further, while only a single machine is illustrated, the term “machine” may also include any collection of machines that individually or jointly execute a set (or multiple sets) of instructions to perform any one or more of the methods discussed herein.

The example computing device 700 includes a processing device (e.g., a processor) 702, a main memory 704 (e.g., read-only memory (ROM), flash memory, dynamic random access memory (DRAM) such as synchronous DRAM (SDRAM)), a static memory 706 (e.g., flash memory, static random access memory (SRAM)) and a data storage device 716, which communicate with each other via a bus 708.

Processing device 702 represents one or more general-purpose processing devices such as a microprocessor, central processing unit, or the like. More particularly, the processing device 702 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 702 may also include one or more special-purpose processing devices such as an application specific integrated circuit (ASIC), a field programmable gate array (FPGA), a digital signal processor (DSP), network processor, or the like. The processing device 702 is configured to execute instructions 726 for performing the operations and steps discussed herein.

The computing device 700 may further include a network interface device 722 which may communicate with a network 718. The computing device 700 also may include a display device 710 (e.g., a liquid crystal display (LCD) or a cathode ray tube (CRT)), an alphanumeric input device 712 (e.g., a keyboard), a cursor control device 714 (e.g., a mouse) and a signal generation device 720 (e.g., a speaker). In at least one embodiment, the display device 710, the alphanumeric input device 712, and the cursor control device 714 may be combined into a single component or device (e.g., an LCD touch screen).

The data storage device 716 may include a computer-readable storage medium 724 on which is stored one or more sets of instructions 726 embodying any one or more of the methods or functions described herein. The instructions 726 may also reside, completely or at least partially, within the main memory 704 and/or within the processing device 702 during execution thereof by the computing device 700, the main memory 704 and the processing device 702 also constituting computer-readable media. The instructions may further be transmitted or received over a network 718 via the network interface device 722.

While the computer-readable storage medium 726 is shown in an example embodiment to be a single medium, the term “computer-readable storage medium” may include a single medium or multiple media (e.g., a centralized or distributed database and/or associated caches and servers) that store the one or more sets of instructions. The term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media.

Improvements Resulting from the Disclosed Embodiments

The disclosed embodiments are implemented by use of a particular machine, and therefore relate to a specific improvement to that machine. In particular, the disclosed embodiments use biosensors and other sensors to detect a variety of emotions and stress levels for a user. Furthermore, biosensors and other sensors detect a variety of emotions with a very low latency. Such biosensors and other sensors are implemented in a device associated with a user, for example, such as a small, wearable device. Such devices may detect biomarkers such as heart and respiration rate, and may include an accelerometer.

A variety of biomarkers may be detected by the biosensors other sensors, such as heart rate variability, variation of cardiac parameters due to respiratory sinus arrhythmia (RSA), change in skin conductance (e.g., galvanic skin response), and/or energy in various EEG bands (such as an alpha band). In addition, the disclosed embodiments may measure the entire range of emotions described and represented in the circumplex model of emotion (see FIG. 1). In particular, the disclosed embodiments may determine which specific emotion and intensity of the emotion associated with the user based on the digital signals derived from the sensors in response to changes in biological process and/or biomarkers. Such aspects can be especially useful in embodiments where students may use the application to seek help from peers, teachers, and/or others. Furthermore, the application may detect if a student is struggling or needs help and co-relating that with the relevant context.

The application may also be used to improve feedback for sports skills. In particular, the application may be used to correct and improve form and technique of athletes performing an athletic move. For example, the application may use signals from accelerometers to correct and improve form and technique of an athlete engaged in an athletic move. In some embodiments, very short snippets of an athletic move may be obtained by the sensors and/or accelerometer, for example, using millisecond accuracy with components identified in all three dimensions (X, Y, Z) of movement. The accelerometer may detect location, movement, speed, and/or other components of the user. The application may then compare these components to the corresponding components of a desired or ideal form. Such aspects may be used to provide recommendations for improvement or otherwise improving the user. Such precise information may not be obtained by a human and may not practicably be calculated by a human, for example, using a pen and paper-based calculations. Furthermore, the suggestions for improvement may be highly nuanced and almost impossible to be captured linguistically.

In addition, the disclosed embodiments may explicitly detect and identify the individual components of a user's context, psychosomatic state, and its variation along regularly sampled time. Such aspects permit the application to create a protocol and procedure to provide improved feedback and assistance to users that was not previously possible. Such aspects may significantly improve the process of co-relating response of one user with another user, with a context dependent psychosomatic state of the second user via the user's biomarkers. Such biomarkers may indicate emotion, thereby enabling the first user to better understand the emotions of the second user, which leads to a variety of helpful processes due to the ability to provide insights that were not efficiently available previously (say, over the Internet). This, in turn, may improve the learning process and/or improve homework completion, for example, electronically.

With respect to sports skills, the disclosed embodiments improve the practice process of an athlete by using combinations of psychosomatic states, energy (e.g., measures of movement) and its rate of dissipation along various dimensions of movements, and form (which may be mined using machine learning), and can greatly enhance the performance of an athlete. Such configurations may also greatly improve practice sessions because coaches are able to provide precise and directed feedback allowing the athletes to improve their performance.

Such aspects may also be applied to public speaking to materially improve the practice of preparation for an important event by providing tools for co-relating emotion of a speaker and a listener, to improve a speaking-heavy but impact sensitive process.

The disclosed embodiment also may improve the efficiency of systems for providing assistance and feedback. In particular, the disclosed embodiments utilize biosensors and biosensing to increase the efficiency of reaching out to individuals in order to ask for help as well as shorten the process of learning from a real-life teacher or coach. In particular, by reading or sensing the emotions of the user using biosensors, the disclosed embodiments are able to provide highly nuanced or intricately detailed expert feedback to the user for various tasks that range from interviews and speeches to sports.

In addition, the disclosed embodiments may allow the user to either complete their homework or complete it more efficiently by displaying the user's psychosomatic state and work at hand (extent of the homework completion or barriers to understanding) to teachers and other students. This may thereby encourage others to help the user if needed, thereby helping to erase the stigma around asking for help. Overall, the disclosed embodiments increase the efficiency of completing and improving activities such as sports, homework, and performances by eliminating the need for a real-life coach. It also helps in reducing anxiety and stress levels for users by providing immediate means for help and assistance with tasks.

In addition, the disclosed embodiments allow a computer to perform a function not previously performable by a computer. In particular, the disclosed embodiments permit feedback and assistance for users to be improved by implementing biosensors to identify user's emotions while performing various task. Furthermore, the disclosed embodiments may explicitly detect and identify the individual components of user's context, psychosomatic state, and its variation along regularly sampled time, which allow various types of monitoring (for example, by parents or remotely situated instructors or therapists). Such aspects were not previously performable by a computer without the features of the disclosed embodiments.

Terms used herein and especially in the appended claims (e.g., bodies of the appended claims) are generally intended as “open” terms (e.g., the term “including” may be interpreted as “including, but not limited to,” the term “having” may be interpreted as “having at least,” the term “includes” may be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases may not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” may be interpreted to mean “at least one” or “one or more”); the same holds true for the use of definite articles used to introduce claim recitations.

In addition, even if a specific number of an introduced claim recitation is explicitly recited, those skilled in the art will recognize that such recitation may be interpreted to mean at least the recited number (e.g., the bare recitation of “two recitations,” without other modifiers, means at least two recitations, or two or more recitations). Further, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, may be understood to contemplate the possibilities of including one of the terms, either of the terms, or both terms. For example, the phrase “A or B” may be understood to include the possibilities of “A” or “B” or “A and B.”

Embodiments described herein may be implemented using computer-readable media for carrying or having computer-executable instructions or data structures stored thereon. Such computer-readable media may be any available media that may be accessed by a general purpose or special purpose computer. By way of example, and not limitation, such computer-readable media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general purpose or special purpose computer. Combinations of the above may also be included within the scope of computer-readable media.

Computer-executable instructions may include, for example, instructions and data which cause a general purpose computer, special purpose computer, or special purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the terms “module” or “component” may refer to specific hardware implementations configured to perform the operations of the module or component and/or software objects or software routines that may be stored on and/or executed by general purpose hardware (e.g., computer-readable media, processing devices, etc.) of the computing system. In some embodiments, the different components, modules, engines, and services described herein may be implemented as objects or processes that execute on the computing system (e.g., as separate threads). While some of the system and methods described herein are generally described as being implemented in software (stored on and/or executed by general purpose hardware), specific hardware implementations or a combination of software and specific hardware implementations are also possible and contemplated. In this description, a “computing entity” may be any computing system as previously defined herein, or any module or combination of modulates running on a computing system.

By the term “substantially” it is meant that the recited characteristic, parameter, or value need not be achieved exactly, but that deviations or variations, including for example, tolerances, measurement error, measurement accuracy limitations and other factors known to those skilled in the art, may occur in amounts that do not preclude the effect the characteristic was intended to provide.

For the processes and/or methods disclosed, the functions performed in the processes and methods may be implemented in differing order as may be indicated by context. Furthermore, the outlined steps and operations are only provided as examples, and some of the steps and operations may be optional, combined into fewer steps and operations, or expanded into additional steps and operations.

This disclosure may sometimes illustrate different components contained within, or connected with, different other components. Such depicted architectures are merely exemplary, and many other architectures can be implemented which achieve the same or similar functionality.

Aspects of the present disclosure may be embodied in other forms without departing from its spirit or essential characteristics. The described aspects are to be considered in all respects illustrative and not restrictive. The claimed subject matter is indicated by the appended claims rather than by the foregoing description. All changes which come within the meaning and range of equivalency of the claims are to be embraced within their scope.

What is claimed is:

1. A method comprising:
detecting a biomarker of a first user using a biosensor associated with the first user;
determining a psychophysiological state of the first user based on the biomarker detected by the biosensor; and
providing feedback to the user via a user interface of a social networking application that links a set of users including the first user to improve performance of the first user in executing a task based on pre-defined metrics, the pre-defined metrics known to the users of the linked set of users to provide feedback or assistance using the social networking application.

2. The method of claim 1, wherein the task includes completing a homework assignment, engaging in a sports activity, public speaking, interviewing or acting.

3. The method of claim 1, wherein the biosensor is included in a wearable network-connected device associated with the first user.

4. The method of claim 1, wherein the biomarker comprises heart rate or respiration of the first user.

5. The method of claim 1, further comprising analyzing the biomarker using non-linear analysis, a PNNx technique, a Root Mean Square of the Successive Differences (RMSSD) technique or Fast Fourier Transforms of respiration.

6. The method of claim 1, further comprising determining a gap between the psychophysiological state of the first user and a desired psychophysiological state.

7. The method of claim 1, wherein the task performed by the first user is a homework assignment, further comprising continuously monitoring stress levels of the first user during performance of the homework assignment using the biomarker detected by the biosensor.

8. The method of claim 7, further comprising displaying a stress level or a level of completion of the homework assignment corresponding to the first user, to a second user using a second user interface of the application.

9. The method of claim 7, further comprising displaying one or more of:
availability to help of a second user to the first user via the user interface of the application;
comprehension of a topic of the first user to a second user via a second user interface of the application;
a suggestion of a study partner based on study habits and patterns; and
a grade impact scale indicating an impact of the homework assignment on an overall grade of the user.

10. The method of claim 1, wherein the task performed by the first user is a sports technique, further comprising:
detecting a position and a movement of a portion of the first user's body using a sensor;
obtaining a desired position and desired movement corresponding to the sports technique; and
comparing the desired position and the desired movement with the detected position and the detected movement of the portion of the first user's body.

11. The method of claim 10, wherein the desired position and desired movement corresponds to a position and a movement of a portion of a professional sports athlete's body.

12. The method of claim 10, wherein providing feedback to the first user is based on differences between the desired position and the desired movement with the detected position and the detected movement of the portion of the first user's body.

13. The method of claim 10, further comprising:
analyzing a mindset of the first user based on the biomarker of the first user;
analyzing comprehension of feedback received by the first user; and
displaying the comprehension or the mindset of the first user to a second user via a second user interface of the application.

14. The method of claim 1, wherein the task performed by the first user is an interview, further comprising:
detecting a set of characteristics of the first user performing the interview; and
comparing the detected set of characteristics with a desired set of characteristics;
wherein the set of characteristics includes one or more of: emotions of the first user, posture of the first user, tone of the first user, and content of the first user's responses.

15. The method of claim 14, wherein providing feedback to the first user is based on differences between the detected set of characteristics and the desired set of characteristics of the interview.

16. The method of claim 1, wherein the task performed by the first user is a speech, further comprising:
obtaining text of the speech including annotations corresponding to desired emotions to be emoted in the speech;
obtaining a video of the user performing the speech using a camera associated with the first user;
detecting a set of characteristics of the first user performing the speech; and
comparing the detected set of characteristics with a desired set of characteristics;
wherein the set of characteristics includes one or more of: emotions of the first user, tone of the first user, hand gestures of the first user, and body language of the first user.

17. The method of claim 16, wherein providing feedback to the first user is based on differences between the detected set of characteristics and the desired set of characteristics of the speech.

18. The method of claim 1, wherein the task performed by the first user is a theater scene, further comprising:
obtaining script of the theater scene including annotations corresponding to desired emotions to be emoted in the theater scene;
obtaining a video of the user performing the theater scene using a camera associated with the first user;
detecting a set of characteristics of the first user performing the theater scene; and
comparing the detected set of characteristics with a desired set of characteristics;
wherein the set of characteristics includes one or more of: emotions of the first user, tone of the first user, hand gestures of the first user, and body language of the first user.

19. The method of claim 18, wherein providing feedback to the first user is based on differences between the detected set of characteristics and the desired set of characteristics of the theater scene.

20. The method of claim 1, wherein the psychophysiological states correspond to a diagram of a circumplex model of emotion.
